(19) United States
(12) Patent Application Publication
(10) Pub. No.: US 2014/0136209 A1
(43) Pub. Date: May 15, 2014

(54) VOICE-ASSISTED BIOMEDICAL MEASUREMENT APPARATUS

(71) Applicant: HEALTH & LIFE CO., LTD., TAIPEI (TW)

(72) Inventors: MENG-YI LIN, TAIPEI (TW); SHAO-HUNG LEE, TAIPEI (TW)

(73) Assignee: HEALTH & LIFE CO., LTD., TAIPEI (TW)

(21) Appl. No.: 13/895,506

(22) Filed: May 16, 2013

(30) Foreign Application Priority Data
Nov. 9, 2012 (TW) 101141847

(51) Int. Cl. A61B 5/00

(52) U.S. Cl. CPC A61B 5/7415
USPC 704/271

(57) ABSTRACT

A voice-assisted biomedical measurement apparatus is revealed. The voice-assisted biomedical measurement apparatus allows users to get measurement results of biological signals and other assistant information in aural form. The voice-assisted biomedical measurement apparatus consists of a sensing unit, a control unit, a voice module and a speaker. The voice-assisted biomedical measurement apparatus further includes a display unit, an operation unit, a memory unit and a data transmission unit. The voice-assisted biomedical measurement apparatus features on that the voice module combines at least one first voice data to form a sentence according to at least one first grammar data when a first control signal is sent from the control unit to the voice module. Moreover, the voice-assisted biomedical measurement apparatus gets at least one second voice data and at least one second grammar data from an external data system through a data transmission unit.

### BACKGROUND OF THE INVENTION

1. Fields of the Invention

The present invention relates to a biomedical measurement apparatus, especially to a voice-assisted biomedical measurement apparatus that combines voice data and grammar data to form sentences. Moreover, the voice-assisted biomedical measurement apparatus also adds or changes voice data and grammar data stored therein through a data transmission unit.

2. Descriptions of Related Art

Biomedical measurement apparatus used by individuals or families outside medical institutes are for monitoring people or patient's physiological conditions at home. Most of these apparatus are easy for carrying and with small volume. Thus the size of the screen used in the small-volume biomedical measurement apparatus is limited. For elders and people with poor eyesight, it's hard to read the information on such small-sized screen.

Thus there is a biomedical measurement apparatus with a voice-assisted function available on the market. During the operation process, the apparatus provides users voice assistance or a measurement result is reported by voice. There is no need for users to read content on the small-sized screen. They can get the data required in aural form. For example, refer to Taiwanese Pat. App. Pub. No. 200528715, a medical diagnostic testing device with voice message capability is revealed. A medical diagnostic testing device is provided with a voice message capability to record one or more voice messages associated with a test result. When a stored test result is retrieved and displayed, the medical diagnostic testing device also plays the one or more recorded voice messages associated with the stored test result.

Refer to Taiwanese App. Pub. No. 200637525, a medical produce with a voice message capability is disclosed. A voice device is built-in or connected outside a medical testing device. After performing tests, the medical testing device sends an encoded data signal to the voice device. Then the data signal is decoded by the voice device so as to output a testing result in voice form. Thus functionality and convenience of the medical product are improved.

Refer to Taiwanese Pat. App. Pub. M403654, an expandable voice capacity microprocessor for biomedical testers is revealed. The expandable voice capacity microprocessor features on that a driver module is disposed in a data processing element and a sound source carrier is removable. When there is a need to increase or decrease voice files, only the sound source carrier stored with the voice file required therein needs to be replaced. There is no need to change the driver module and the sound source carrier simultaneously. Thus the cost is saved and the processing steps are reduced.

The above three patents all relate to a voice-assisted biomedical measurement apparatus. A measuring result of the apparatus is presented by a voice device arranged in the apparatus. Thus users get the information by hearing. However, the language of the previous two voice devices is unable to be changed. Although the last voice device is expandable or the language of the voice is changeable by replacement of the sound source carrier, it's still inconvenient in use due to the replacement of the sound source carrier. Moreover, the voice of conventional voice-assisted medical measurement apparatus is composed of sentences recorded in advance. Thus the storage space of the voice data is difficult to be reduced and the apparatus is unable to support sentences with more complicated structure.

In order to solve the above shortcomings, a voice-assisted biomedical measurement apparatus is provided. The voice-assisted biomedical measurement apparatus stores at least one first voice data and at least one first grammar data and includes a voice module that retrieves the first voice data required and combines the first voice data to form a sentence according to the first grammar data. Then a speaker is driven to output the sentence. Moreover, the present invention gets at least one second voice data and at least one second grammar data from an external data system through a data transmission unit. There is no need to replace hardware for changing the language of the voice that meets the user's requirements. Thus the apparatus is more convenient in use.

A voice-assisted medical measurement apparatus as presented in the invention can audibly present the biomedical information and help users get the measuring result, a guidance or other additional information. The voice-assisted biomedical measurement apparatus is composed of a sensing unit, a control unit, a voice module and a speaker. The voice-assisted biomedical measurement apparatus further includes a display unit, an operation unit, a memory unit and a data transmission unit. The apparatus features on that the control unit sends a first control signal to make the voice module retrieve at least one first voice data and at least one first grammar data after the control unit receiving a biological signal from the sensing unit. Then the voice module combines the first voice data to form a sentence according to the first grammar data. Moreover, the apparatus gets at least one second voice data and at least one second grammar data from an external data system through the data transmission unit so as to increase or change the voice data and the grammar data stored therein.

### SUMMARY OF THE INVENTION

Therefore it is a primary object of the present invention to provide a voice-assisted biomedical measurement apparatus that includes a voice module that combines a first voice data to form a sentence according to a first grammar data in response to a first control signal from a control unit. Then the sentence is audibly outputted by a speaker so as to help elders or people with poor eyesight learn a measurement result of a biological signal and related assistant information.

It is another object of the present invention to provide a voice-assisted medical measurement apparatus that retrieves at least one second voice data and at least one second grammar data from an external data system through a data transmission unit. Then the second voice data and the second grammar data are added into a memory unit or used to replace a first voice data and a first grammar data stored in the memory unit. Therefore the language of the sentence can be changed according to user's needs. There is no need to store voice data and grammar data of multiple languages in the memory unit in advance.

It is a further object of the present invention to provide a voice-assisted medical measurement apparatus in which a biological signal detected by a sensing unit is converted to a piece of biological data by a control unit and then the biological data is stored in a memory unit. The memory unit is also used to store the first voice data, the first grammar data, the second voice data and the second grammar data.

In order to achieve the above objects, a voice-assisted biomedical measurement apparatus of the present invention is disposed with a sensing unit to detect biological signals and transmit the biological signals to a control unit connected to the sensing unit. The control unit can receive the biological signal or an operation signal produced by an operation unit, and then generates a first control signal corresponding to the biological signal or the operation signal for control of a voice module connected to the control unit. Thus the voice module combines at least one first voice data to form a sentence according to at least one first grammar data and in response to the first control signal. A speaker is also driven to output the sentence and provide users a measurement result of the biological signal and related assistant information. The present invention further includes an operation unit connected to the control unit. The user can transmit operation signals to the control unit by the operation unit while operating the apparatus. Moreover, a display unit is connected to the control unit and used for receiving a second control signal generated by the control unit according to the biological signal or the operation signal. Then a measurement result of the biological signal and assistant information related to the biological signal in visual form are output according to the second control signal. Furthermore, the present invention further includes a memory unit included in the voice module or independent from the voice module. The memory unit is connected to the control unit and a voice unit in the voice module/or the voice module. The memory unit is used to store at least one first voice data, at least one grammar data, and a biological data obtained from the biological signal being converted by the control unit. The present invention further includes a data transmission unit connected to the apparatus and used as an interface for data exchange between the apparatus and an external data system. Thus the apparatus can get at least one second voice data and at least one second grammar data from the external data system so as to add the second voice data and the second grammar data in a memory unit or use the second voice data and the second grammar data to replace the first voice data and the first grammar data in the memory unit. The biological data stored in the memory unit is able to be transmitted to the external data system.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic drawing showing connection of components of an embodiment according to the present invention;

FIG. 2 is a schematic drawing showing structure of an embodiment according to the present invention;

FIG. 3 is a schematic drawing showing connection of components of another embodiment according to the present invention;

FIG. 4 is a schematic drawing showing structure of another embodiment according to the present invention;

FIG. 5 is another schematic drawing showing structure of the embodiment in FIG. 4 according to the present invention;

FIG. 6 is a schematic drawing showing connection of components of a further embodiment according to the present invention;

FIG. 7 is a schematic drawing showing connection of components of a further embodiment according to the present invention.

### DETAILED DESCRIPTION OF THE PREFERRED EMBODIMENT

A voice-assisted biomedical measurement apparatus of the present invention features on that: a control unit receives a biological signal through a sensing unit or receives an operation signal through an operation unit, and then generates a first control signal according to the biological signal or the operation signal. A voice module connected to the control unit retrieves at least one first voice data and at least one first grammar data in response to the first control signal and combines them to form a sentence. Then the sentence is output by a speaker. Thus a plurality of sentences could be produced by combination with the first voice data and the first grammar data. That will make the biomedical apparatus capable to present audio content with more complex meaning and save the memory space. Moreover, the apparatus is connected to an external data system by a data transmission unit to get at least one second voice data and at least one second grammar data from the external data system. Then the second voice data and the second grammar data are added in a memory unit or used to replace the first voice data and the first grammar data stored in the memory unit. Thus the language of the sentence can be changed flexibly according to user's needs. There is no need to save voice data and grammar data of multiple languages in the memory unit in advance so that the space for data storage is saved. Besides storage of the first voice data, the first grammar data, the second voice data, and the second grammar data, the memory unit also stores a biological data obtained by the biological signal being converted by the control unit, and transmits the biological data to an external data system through the data transmission unit.

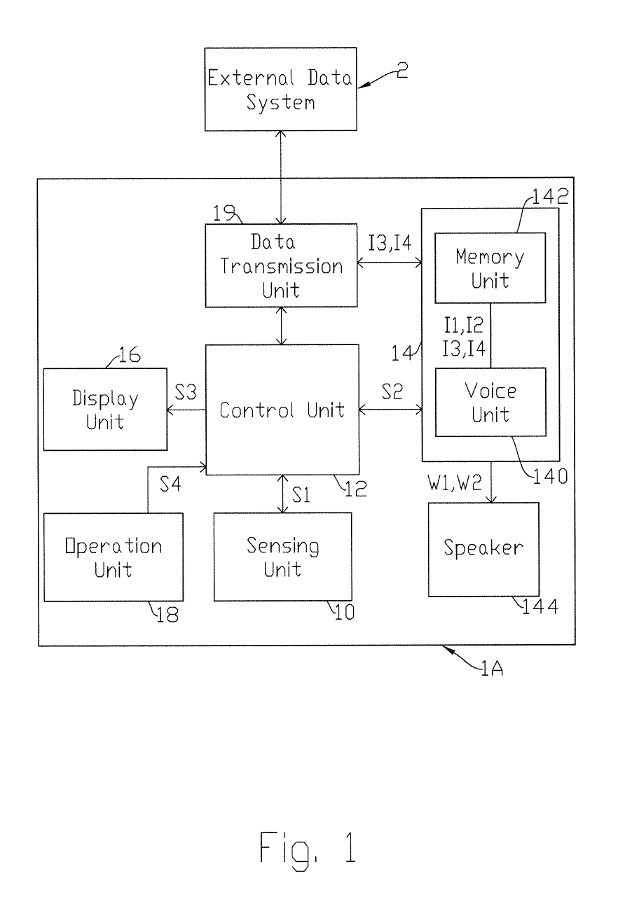

Refer to FIG. 1, a voice-assisted biomedical measurement apparatus 1A of the present invention includes a control unit 12 as a center that connects other components. The control unit 12 is connected to a sensing unit 10, a voice module 14, a display unit 16, an operation unit 18 and a data transmission unit 19 respectively. The voice module 14 consists of a voice unit 140 and a memory unit 142 that are respectively connected to a speaker 144 and the data transmission unit 19. The voice-assisted biomedical measurement apparatus 1A is connected to an external data system 2 by the data transmission unit 19.

While detecting biological signals, the sensing unit 10 is used for detection and the operation unit 18 is used to set sensing conditions or confirm sensing motion. After receiving a biological signal S1 detected by the sensing unit 10 and an operation signal S4 transmitted from the operation unit 18, the control unit 12 generates a first control signal S2 and a second control signal S3 correspondingly according to the biological signal S1 or the operation signal S4. The first control signal S2 and the second control signal S3 are respectively transmitted to the voice module 14 and the display unit 16. After receiving the second control signal S3, the display unit 16 shows a measurement result of the biological signal S1 and auxiliary information related to the biological signal S1 in words or figures. After the voice module 14 receiving the first control signal S2, a first voice data I1 and a first grammar data 12 stored in the memory unit 142 are retrieved and then the voice unit 140 combines the first voice data 11 and the first grammar data 12 to form a sentence W1 according to the first control signal S2. Then the speaker 144 is driven to output the sentence W1 and the measurement result of the biological signal S1 is displayed. Moreover, the voice unit 140 can also combine the data to form an auxiliary sentence W2. The auxiliary sentence W2 is a warning or a suggestion corresponding to the biological signal S1.

When the user needs to use language different from that of the first voice data 11 and the first grammar data 12, the voice-assisted biomedical measurement apparatus 1A of the present invention is connected to an external data system 2 by the data transmission unit 19. The data transmission unit 19 is connected to the external data system 2 by, but not limited to, an USB interface, an UART interface, a bluetooth interface, a RFID/NFC interface, a WiFi interface, or a GPRS interface. The external data system 2 includes, but not limited to, a personal computer, a smart phone, a network server or a mobile storage device. The user can get a second voice data 13 and a second grammar data 14 from the external data system 2. Then the second voice data 13 and the second grammar data 14 are transmitted to the memory unit 142 by the data transmission unit 19. At this moment, the user can choose whether to save the first voice data 11 and the first grammar data 12 or not so as to achieve voice and grammar data addition or replacement. After getting the second voice data 13 and the second grammar data 14, the voice unit 140 combines the second voice data 13 and the second grammar data 14 to form a sentence of the language required. Next the voice of the language is output by the speaker 144. Thus the present invention language of the voice can be changed flexibly according to users' needs and there is no need to need to store voice data of various languages and grammar data of various languages in the memory unit 142 in advance. Moreover, the first voice data 11 and the first grammar data 12 are replaced by the second voice data 13 and the second grammar data 14 respectively after the second voice data 13 and the second grammar data 14 being downloaded. Thus the storage space occupied by the voice data and the grammar data is saved. The manufacturers and dealers of the present invention can adjust the voice data and the grammar data in the memory unit 142 in advance according to languages used on the markets where the products are sold.

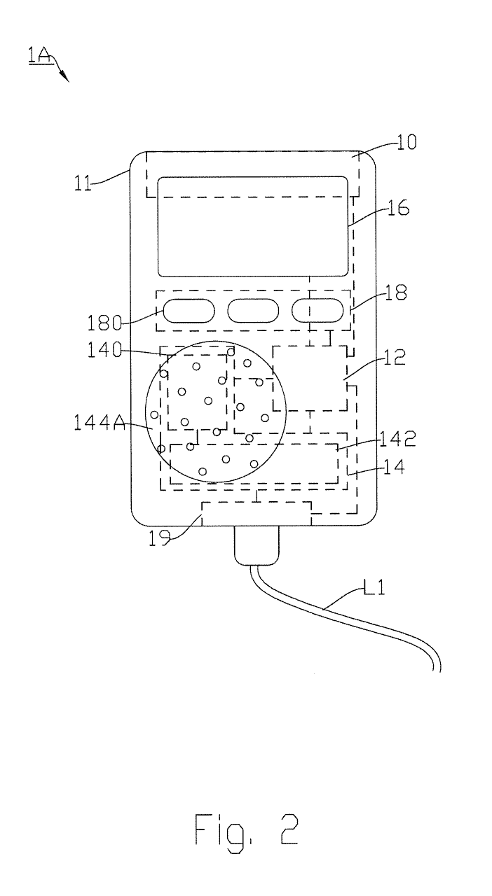

Refer to FIG. 2, a schematic drawing showing structure of an embodiment of the present invention is revealed. As shown in the figure, a voice-assisted biomedical measurement apparatus 1A of the present invention includes a sensing unit 10, a control unit 12, a voice unit 140 included in the voice module 14, a memory unit 142 of the voice module 14, a speaker 144A driven by the voice module 14, a display unit 16 driven by the control unit 12, an operation unit 18 and a data transmission unit 19. The sensing unit 10, the control unit 12, the voice module 14, and the data transmission unit 19 are set in a housing 11. The display unit 16 is disposed on the surface of the housing 11. The housing 11 is also arranged with the operation unit 18. The operation unit 18 consists of a plurality of operation buttons 180. The data transmission unit 19 is connected to an external data system (not shown in the figure) outside the housing 11.

By the sensing unit 10, the control unit 12, the voice module 14, and the operation unit 18 being packaged and connected to the housing 11, the present invention is easy to be produced, transported, sold and used. The voice-assisted biomedical measurement apparatus 1A is applied to detect a single kind or multiple kinds of biological signals by the sensing unit 10 used in combination with a program design of the control unit 12. Moreover, in this embodiment, the built-in speaker 144 is embedded in the surface of the housing. There is no need to connect the biomedical measurement apparatus 1A with other speakers and this is convenient for carrying.

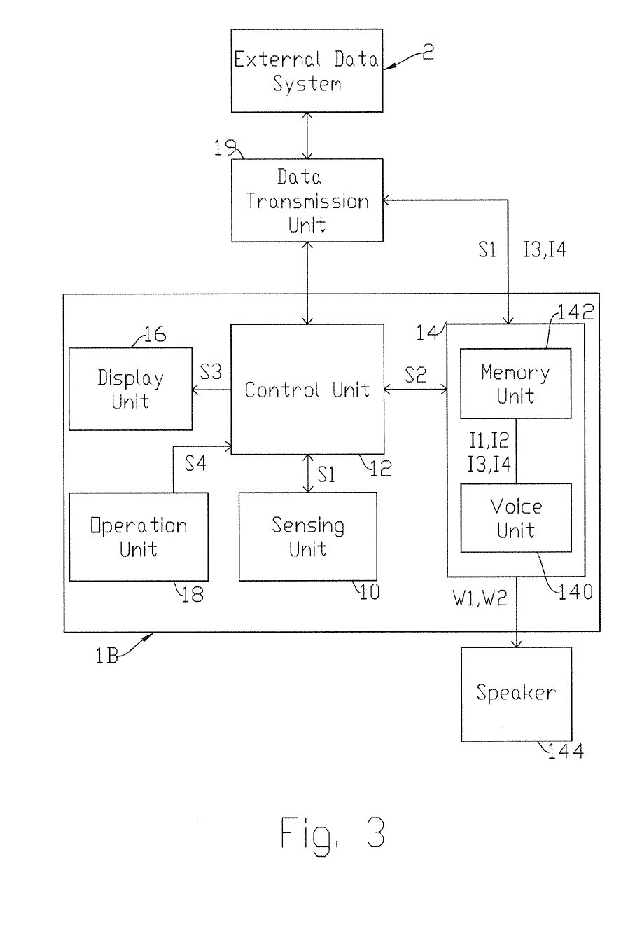

Refer to FIG. 3, a schematic drawing showing connection of components of another embodiment according to the present invention is disclosed. The difference between this embodiment and the above one is in that both a speaker 144 and a data transmission unit 19 are disposed outside the voice-assisted biomedical measurement apparatus 1B and are pluggable and replaceable external components. As to the data transmission unit 19, it includes at least one transmission interface so as to be applied to various external data systems.

This embodiment has the advantage that the speaker 144 and the data transmission unit 19 are replaceable. Thus users can choose earphones or an external speaker as the speaker 144 and change the data transmission unit 19 with different transmission surface such as an USB interface, an UART interface, a bluetooth interface, a RFID/NFC interface, a WiFi interface, or a GPRS interface. Thus the voice-assisted biomedical measurement apparatus 1B can be applied to various external data systems.

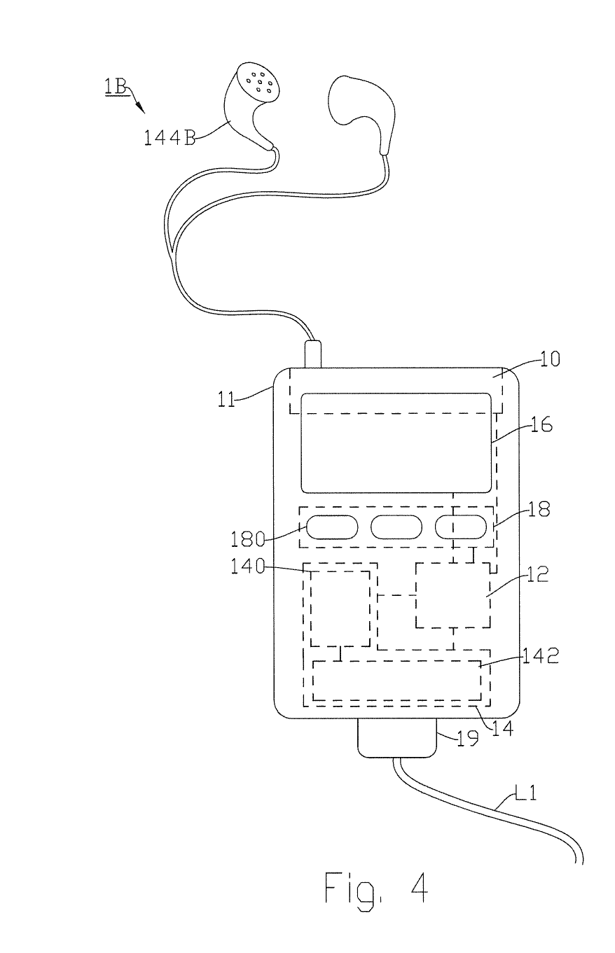

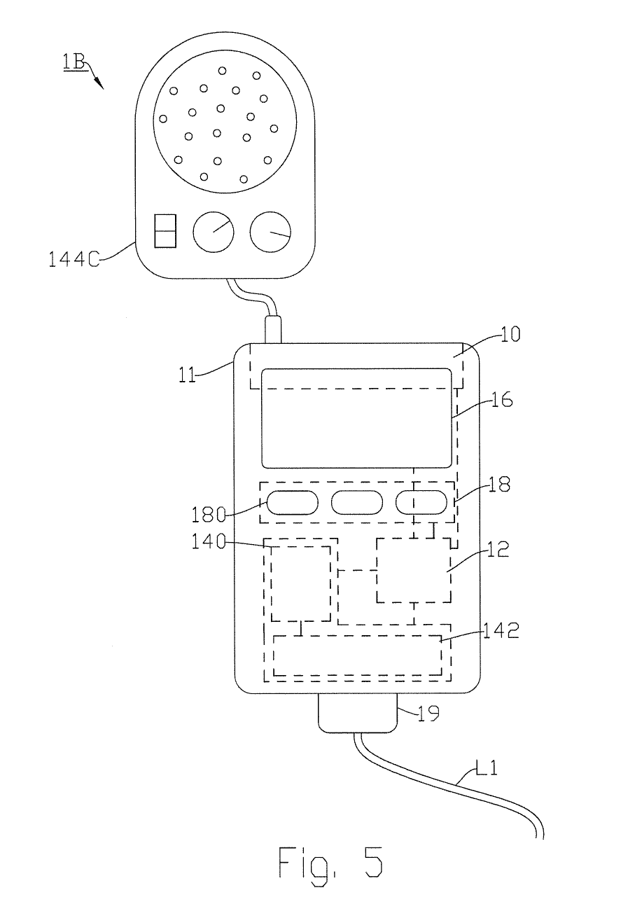

Refer to FIG. 4 and FIG. 5, schematic drawings showing structure of another embodiment according to the present invention are revealed. A voice-assisted biomedical measurement apparatus 1B of this embodiment according to the present invention includes a sensing unit 10, a control unit 12, a voice unit 140 included in the voice module 14, a memory unit 142 of the voice module 14, a display unit 16 driven by the control unit 12, and an operation unit 18. The sensing unit 10, the control unit 12, and the voice module 14 are arranged in a housing 11. The display unit 16 is disposed on the surface of the housing 11. The housing 11 is also set with the operation unit 18. The operation unit 18 consists of a plurality of operation buttons 180. A speaker 144B or 144C driven by the voice module 14 is arranged outside the housing 11 and is connected to the voice module 14. Moreover, a data transmission unit 19 is connected to the housing 11 and also coupled to the control unit 12 and the memory unit 142. By a cable L1, the data transmission unit 19 is connected to an external data system (not shown in the figure).

By the sensing unit 10, the control unit 12, the voice module 14, and the operation unit 18 being packaged and connected to the housing 11, the present invention is easy to be produced, transported, sold and used. The voice-assisted biomedical measurement apparatus 1B is applied to detect a single kind or multiple kinds of biological signals by the sensing unit 10 used together with a program design of the control unit 12.

As shown in the FIG. 4, the speaker is an earphone 144B. The advantage of the earphone 144B is in that the user can listen to voice data by using the earphone 144B when there are some other people come up so as to prevent interfering with others or privacy leakage. Moreover, the earphone 144B can be changed into an external speaker 144C. The advantage of the external speaker 144C is in that the volume of the external speaker 144C is easy to adjust. Thus users and other people on the scene such as medical staff and families can also listen to the voice report.

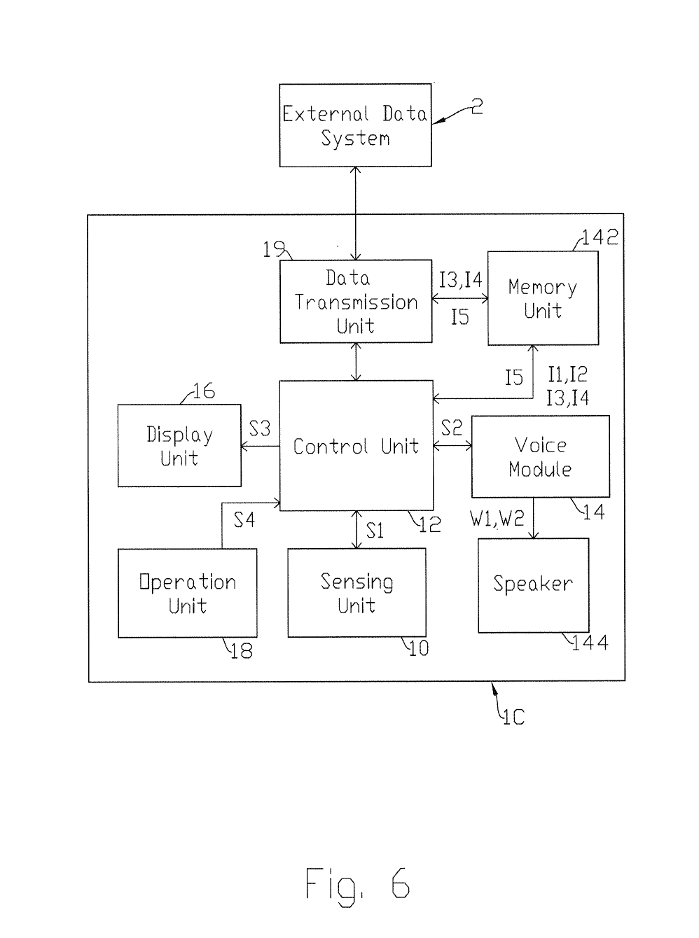

Refer to FIG. 6, a schematic drawing showing connection of components of a further embodiment according to the present invention is revealed. As shown in figure, a voice-assisted biomedical measurement apparatus 1C includes a control unit 12 used as a center and connected to other components. The control unit 12 is connected to a sensing unit 10, a voice module 14, a memory unit 142, a display unit 16, an operation unit 18, and a data transmission unit 19. The voice module 14 is connected to a speaker 144 while the display unit 16 is connected to the data transmission unit 19. The voice-assisted biomedical measurement apparatus 1C is connected to an external data system 2 by the data transmission unit 19.

The difference between the third embodiment and the first embodiment mentioned above is in that the memory unit 142 of this embodiment is not included in the voice module 14. The memory unit 142 is directly connected to the control unit 12 so that the memory unit 142 receives and stores a biological data 15 from the sensing unit 10. The biological data 15 is obtained by a biological signal S1 from the sensing unit 10 being converted by the control unit 12.

When the user intends to send the biological data 15 stored in the memory unit 142 to the external data system 2 for being recorded, the biomedical measurement apparatus 1C is connected to the external data system 2 by the data transmission unit 19. Through the data transmission unit 19, the biological data 15 stored in the memory unit 142 is delivered to the external data system 2. Thus the storage of the biological data 15 is not limited by capacity of the memory unit 142. Thus a plurality pieces of or long-term biological data 15 can be recoded and saved for follow-up or checkup.

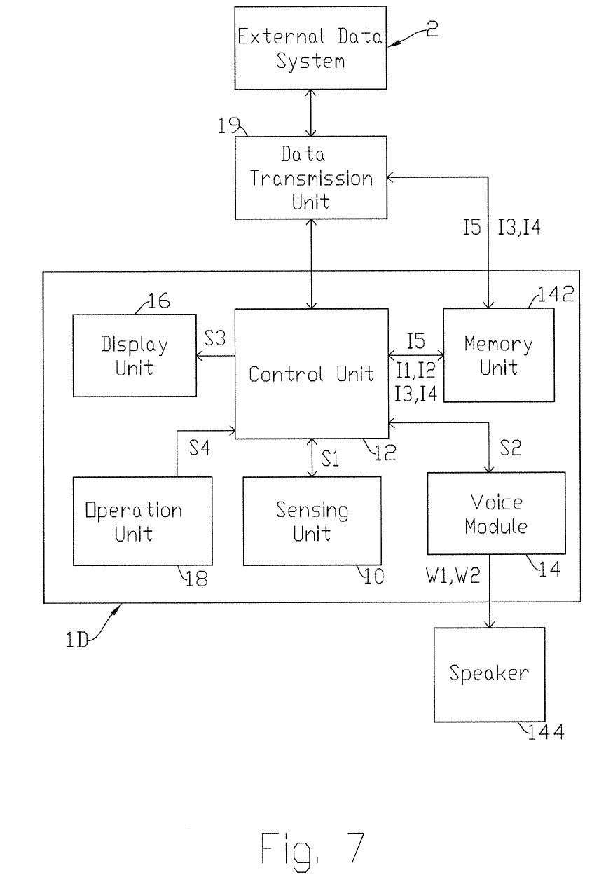

Refer to FIG. 7, a schematic drawing showing connection of components of a fourth embodiment according to the present invention is revealed. The difference between this embodiment and the third embodiment mentioned above is in that this embodiment includes a speaker 144 and a data transmission unit 19 arranged outside a voice-assisted biomedical measurement apparatus 1D. Moreover, the speaker 144 and the data transmission unit 19 are pluggable. The data transmission unit 19 includes at least one transmission interface so as to be applied to various external data systems. Like the second embodiment, the advantage of this embodiment is in that both the speaker 144 and the data transmission unit 19 are replaceable. Various kinds of speakers are provided to be selected by users and various kinds of transmission interfaces suitable for different external data systems are used.

In summary, a voice-assisted biomedical measurement apparatus of the present invention includes a control unit that receives signals from a sensing unit and an operation unit. The control unit also controls a display unit and a voice module to output measurement results of biological signals. Then the results are stored in a memory unit of the voice module. The voice-assisted biomedical measurement apparatus features on that voice data and grammar data used by the voice module can be retrieved from an external data system through a data transmission unit. Then a voice unit of the voice module combines the voice data and the grammar data to form various voice sentences according to the first control signal. Thus not only memory space occupied by the voice and grammar data is saved, the language of the sentence can also be changed flexibly according to user's needs.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A voice-assisted biomedical measurement apparatus comprising:
a sensing unit for detecting a biological signal;
a control unit connected to the sensing unit for receiving the biological signal and generating a first control signal; and
a voice module connected to the control unit, retrieving at least one first voice data and at least one first grammar data according to the first control signal, combining the first voice data with first grammar date to form at least one sentence and driving a speaker to output the sentence.

2. The voice-assisted biomedical measurement apparatus as claimed in claim 1, wherein the control unit is connected to a display unit; the control unit generates a second control signal according to the biological signal; the display unit receives the second control signal and shows a visual information.

3. The voice-assisted biomedical measurement apparatus as claimed in claim 1, wherein the control unit is connected to an operation unit; the operation unit sends an operation signal to the control unit and the control unit generates the first control signal according to the operation signal.

4. The voice-assisted biomedical measurement apparatus as claimed in claim 2, wherein the control unit is connected to an operation unit, the operation unit sends an operation signal to the control unit and the control unit generates the second control signal according to the operation signal.

5. The voice-assisted biomedical measurement apparatus as claimed in claim 1, wherein the voice module includes a voice unit and a memory unit; the voice unit is electrically connected to the memory unit while the memory unit is for storing the first voice data and the first grammar data.

6. The voice-assisted biomedical measurement apparatus as claimed in claim 5, wherein the memory unit is for storing a biological data; the biological data is generated from the biological signal being converted by the control unit.

7. The voice-assisted biomedical measurement apparatus as claimed in claim 1, wherein the control unit is connected to a memory unit; the memory unit is for storing the first voice data and the first grammar data.

8. The voice-assisted biomedical measurement apparatus as claimed in claim 7, wherein the memory unit is for storing a biological data; the biological data is generated from the biological signal being converted by the control unit.

9. The voice-assisted biomedical measurement apparatus as claimed in claim 1, further comprising a data transmission unit which is built-in or connected outside the voice-assisted biomedical measurement apparatus; the data transmission unit is an interface for data exchange between the biomedical measurement apparatus and an external data system.

10. The voice-assisted biomedical measurement apparatus as claimed in claim 9, wherein the voice module gets at least one second voice data and at least one second grammar data from the external data system through the data transmission unit.

11. The voice-assisted biomedical measurement apparatus as claimed in claim 10, wherein the voice module includes a voice unit and a memory unit; the voice unit is electrically connected to the memory unit while the memory unit is for storing the second voice data and the second grammar data.

12. The voice-assisted biomedical measurement as claimed in claim 10, wherein the control unit is connected to a memory unit; the memory unit is for storing the second voice data and the second grammar data.

13. A method for a voice module of a biomedical measurement apparatus to form sentences comprising the steps of:
receiving a first control signal from a control unit by the voice module;
retrieving at least one first voice data according to the first control signal; and
combining the first voice data with at least on grammar data to form at least one sentence.
